(19) United States
(12) Patent Application Publication
(10) Pub. No.: US 2006/0149587 A1
(43) Pub. Date: Jul. 6, 2006

(54) Automated system and method for processing prescriptions

(75) Inventors: Kenneth A. Hill SR., Fort Worth, TX (US); Mark A. Stalzer, Haltom City, TX (US); Sean P. Bloodgood, Fort Worth, TX (US); Todd A. Crosslin, Azle, TX (US); John T. McCray, Granbury, TX (US)

Correspondence Address: FISH & RICHARDSON P.C., P.O. BOX 1022, MINNEAPOLIS, MN, 55440-1022, US

(73) Assignee: PDX, Inc.

(21) Appl. No.: 09/996,100

(22) Filed: Nov. 26, 2001

(51) Int. Cl. G06Q 50/00; A61B 5/00

(52) U.S. Cl. 705/2; 600/300

(57) ABSTRACT

An automated system and method for processing prescription requests is disclosed, whereby a patient or physician enters a prescription request to an automated pharmacy prescription processing system. For example, a request for a new or refill prescription can be transmitted from a physician's office to the prescription processing system as a digital file or facsimile message, or using keypad or voice commands in an Interactive Voice Response (IVR) system running in the prescription processing system. A request for a prescription refill can also be entered by a patient using an IVR system, or the patient can physically carry the refill prescription request to a pharmacy for entry to the prescription processing system by a technician. The automated pharmacy system determines whether the new or refill prescription request can be filled by a central fill inventory. The automated pharmacy prescription processing system sends eligible, pending prescription requests to an automated central fill prescription request processing system. The automated central fill processing system processes and fills each valid prescription request from a central fill inventory, initiates a quality assurance procedure to double-check each prescription to be filled, labels the double-checked, approved prescriptions, and routes them to a staging area for shipment to the appropriate pharmacy stores.

### TECHNICAL FIELD OF THE INVENTION

The present invention relates in general to the pharmacy practice management system and software development fields and, in particular, but not exclusively, to a computer-implemented system and method for processing high-volume prescriptions.

### BACKGROUND OF THE INVENTION

Within the next five years in the pharmaceutical industry, the volume of dispensed prescriptions is expected to increase by about 46 percent. On the other hand, the number of pharmacists available to dispense those prescriptions is expected to increase by only about 3-5 percent. As a result of the rapidly increasing volume of prescriptions being filled, pharmacy professionals are often overworked, and customers' waiting times for prescriptions are increasing significantly. Consequently, a pressing need exists for a high-volume prescription filling system and method that will increase the productivity of pharmacy professionals, and decrease the waiting time for customers' prescriptions to be filled.

Disease management is an evolving aspect of the health management field, which includes the use of information systems, practice guidelines, and case management to improve the long-term or chronic care of patients. Many Health Maintenance Organizations (HMOs) and Preferred Provider Organizations (PPOs) have comprehensive disease management programs in place for such ailments as Alzheimer's, asthma, arthritis, diabetes, congestive heart failure, renal failure, cancer, high-risk pregnancy, and ocular diseases (just to name a few). An important goal of disease management programs is to improve the quality of the long-term patient care and services being provided, and to decrease the costs for providing that level of care and those services.

Certain U.S. federal and local government programs provide no-cost or low-cost drugs for indigent patients requiring long-term care. For example, the Federal Government contracts with pharmaceutical manufacturers for special pricing, 240-B, for government institutions such as VA hospitals and Community Health Centers. These prices are not available to retail pharmacies, and retail pharmacies are not presently allowed to have these inventories mixed with their retail inventories within the confines of their stores. Consequently, a pressing need exists for a prescription processing system which allows pharmacists at a retail pharmacy to perform disease management, drug utilization review, and provide a 2-3 day prescription supply to patients, and then pass the transaction to an off-site robotic facility to fill the remaining drug quantity from another inventory.

### SUMMARY OF THE INVENTION

According to the present invention, problems and disadvantages associated with previous techniques for filling prescriptions are substantially reduced or eliminated.

According to one example embodiment of the present invention, an automated system and method for processing prescription requests is provided, whereby a patient or physician enters a prescription request into an automated pharmacy prescription processing system within a retail store. For example, a request for a new or refill prescription can be transmitted from a physician's office to the prescription processing system within a retail outlet as a digital file or facsimile message, or using keypad or voice commands in an Interactive Voice Response (IVR) system running in the prescription processing system. A request for a prescription refill can also be entered by a patient using an IVR system, or the patient can physically carry the refill prescription request to a pharmacy for entry to the prescription processing system by a technician. The automated pharmacy system determines whether the new or refill prescription request can be filled by a central fill inventory located at a remote site. For example, the automated pharmacy system determines whether the prescription request is eligible to be filled by a central fill inventory, and also whether the prescribed drug is available to be filled by the central fill inventory. The automated pharmacy prescription processing system sends eligible, pending prescription requests to an automated central fill prescription request processing system. The automated central fill processing system processes and fills each valid prescription request from a central fill inventory, initiates a quality assurance procedure to double-check each prescription to be filled, labels the double-checked prescriptions that are approved for filling, and routes the filled prescriptions to a staging area for shipment to the appropriate pharmacy stores, whereby a pharmacist at the store performs a computer-assisted quality assurance check of each prescription.

The present invention provides a number of technical advantages over previous techniques. For example the present invention provides an automated prescription processing system that can efficiently process and fill an extremely large volume of prescription requests from a central fill inventory using robotics. As a result, the productivity of pharmacy professionals can be increased, and customers' waiting times for prescriptions to be filled can be decreased. Also, as a result of using a central fill inventory to fill prescription requests (e.g., in government-funded indigent patient, long-term care programs), the government-funded inventory is not merged in any respect with local pharmacies' inventories. Consequently, the present invention allows pharmaceutical suppliers to maintain a centralized inventory of drugs in a manner that takes full advantage of economies of scale to minimize costs. Through freeing up pharmacists' time by filling prescriptions with high-speed robotics at remote sites, rather than having pharmacists fill them manually, the most important goals of disease management programs can be met: to improve the quality of the long-term patient care and services being provided; and to decrease the costs for providing that level of care and those services.

Other technical advantages of the present invention will be readily apparent to one skilled in the art from the following figures, description and claims.

### BRIEF DESCRIPTION OF THE DRAWINGS

For a more complete understanding of the present invention and its advantages, reference is now made to the following descriptions, taken in conjunction with the accompanying drawings, in which:

FIG. 1 is a block diagram that illustrates an exemplary system and method that can be used to implement one embodiment of the present invention;

FIG. 2 is a flow diagram that illustrates an exemplary method for processing prescriptions, which can be used to implement the embodiment shown in FIG. 1;

FIG. 3 is a flow diagram that illustrates an exemplary method for processing prescription requests, which can be executed by a computer processor in accordance with an embodiment of the present invention;

FIG. 4 is a flow diagram that illustrates an exemplary method which can be used to transmit prescription request information between a pharmacy system and a central fill system, in accordance with an embodiment of the present invention;

FIG. 5 is a flow diagram that illustrates an exemplary computer-implemented method that can be used by a central fill system and a pharmacy system to process prescription requests, in accordance with an embodiment of the present invention;

FIG. 6 is a flow diagram that illustrates an exemplary method that can be implemented by a computer processor associated with a central fill system to process prescription requests, in accordance with an embodiment of the present invention; and

FIG. 7 is a flow diagram that illustrates an exemplary method that can be used by a central fill system to process prescription requests, in accordance with an embodiment of the present invention.

### DETAILED DESCRIPTION OF THE INVENTION

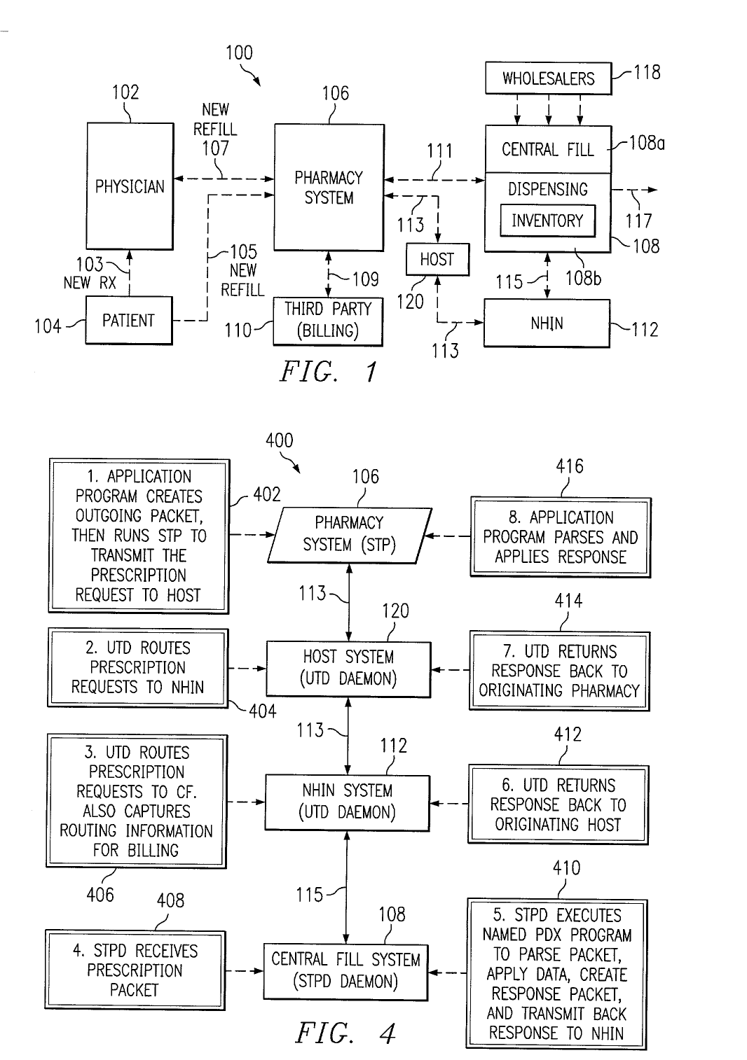

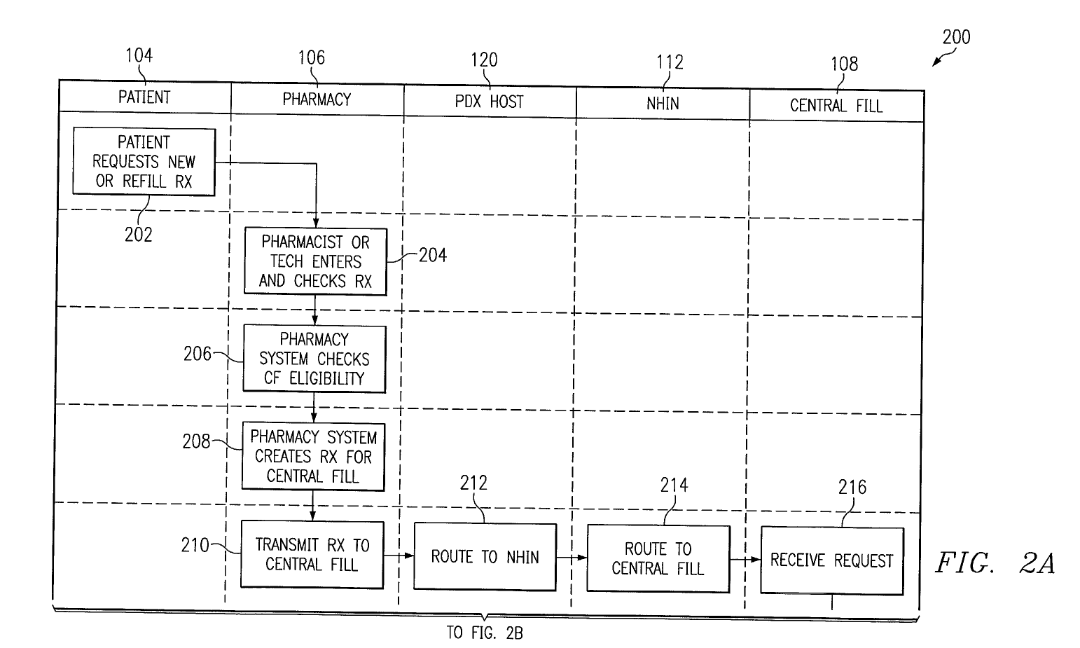

The preferred embodiment of the present invention and its advantages are best understood by referring to FIGS. 1-7 of the drawings, like numerals being used for like and corresponding parts of the various drawings.

FIG. 1 is a block diagram that illustrates an exemplary system and method 100 that can be used to implement one embodiment of the present invention. Essentially, as FIG. 1 shows, a prescription to be filled can be conveyed to a pharmacy in a number of ways. For example, a patient 104 in need of long-term or short-term pharmaceutical care can proceed to a clinic or office of a physician 102 (represented by the dashed line 103). The physician or prescriber 102 can write a prescription with or without refills that the patient 104 can convey to a pharmacy or provider 106 by hand. The function of conveying a written prescription to a pharmacy 106 is represented by the dashed line 105. As an alternative, physician 102 can use an automated system (e.g., RxPad®, etc.) to convey a new prescription via a communication link to an automated prescription management system at pharmacy 106. Preferably, the electronic transmission of prescription information between prescribers and providers is performed in accordance with a current National Council for Prescription Drug Programs (NCPDP) Script Standard (e.g., Script Standard V.1.5). This standard addresses, among other things, the electronic transmission of new prescriptions, prescription refill requests, prescription fill status notifications, and prescription cancellation notifications. The electronic transmission of 071554.0102 prescription information between the prescriber (102) and provider (106) is represented by the dashed line 107.

For the example embodiment illustrated by FIG. 1, pharmacy system 106 is preferably a computer-implemented system for processing prescription requests. As such, the functions of pharmacy system 106 can be implemented by one or more application programs executed by an appropriate computer processor (not explicitly shown). In addition to the methods described above, a prescription can be entered into computer-implemented pharmacy system 106 in a number of different ways. For example, a pharmacy system employee or technician can use a Graphical User Interface (GUI) to enter a new or refill prescription received from a patient 104 (or by telephone from prescriber 102) into pharmacy system 106, by typing in the prescription information to a prescription filling screen (e.g., Multiple Fill Queue screen for refills, or Rx Filling screen for new prescriptions) displayed on a computer monitor. Alternatively, for example, patient 104 can enter a request for a prescription refill via the Internet (e.g., using a browser to access the pharmacy system's web page and enter prescription refill information). Also, as another example, patient 104 can use an IVR system operated by pharmacy system 106 to enter a request for a prescription refill. An IVR system can allow a patient to ask questions and provide answers for pharmacy system 106 by pressing the appropriate keys on a touch-tone phone, or stating certain words such as “yes” or “no”.

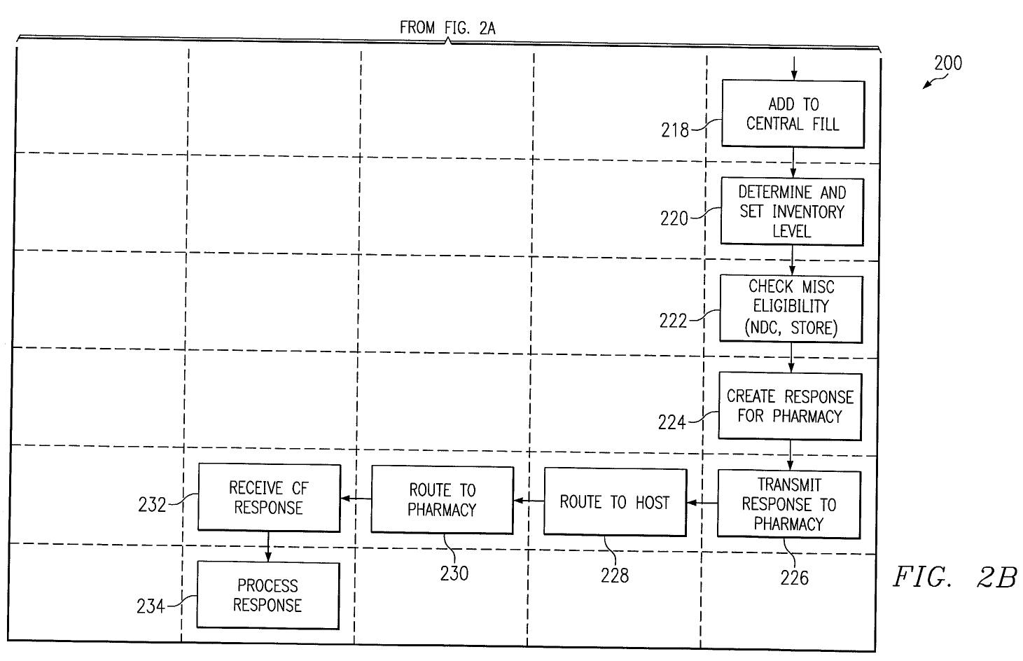

FIG. 2 is a flow diagram that illustrates an exemplary method 200 for processing prescriptions, which can be used to implement an embodiment of the present invention. For example, method 200 can be used for implementing the exemplary embodiment illustrated by FIG. 1. Referring to FIGS. 1 and 2, at step 202, patient 104 presents a request for a new or refill prescription to pharmacy system 106. For this example, pharmacy system 106 can include an individual pharmacy store in a chain of stores or pharmacies. Preferably, as part of the prescription request, patient 104 specifies the date and time when the prescribed drug or drugs are to be picked up. At step 204, a pharmacist or pharmaceutical technician checks the received prescription information for errors, and if no errors or only minor errors (e.g., typos) are found, enters the prescription information to pharmacy system 106.

At step 206, pharmacy system 106 determines whether the prescription to be filled is eligible for processing by central fill system 108. For the example embodiment illustrated by FIG. 1, central fill system 108 is also preferably a computer-implemented system for processing prescription requests. In this regard, the automated functions of central fill system 108 can be implemented by one or more application programs executed by an appropriate computer processor (not explicitly shown). For this example embodiment, in order for a prescription to be eligible for central fill processing, pharmacy system 106 can determine whether the following rules have been followed: (1) The patient has elected to pick up the prescribed drug after the next delivery cycle of central fill system 108; (2) The patient allows the prescription to be filled by central fill system 108; (3) The prescribed drug is listed on the central fill system's formulary (i.e., catalog of products available); (4) The local State government allows drugs to be filled by a central fill facility; and (5) If the request is for a new prescription, the local State government allows new prescriptions to be filled by a central fill facility. If a prescription request follows the above-described rules, for this embodiment, pharmacy system 106 performs edit checks on the entered prescription information, and also a Drug Utilization Review (DUR) for the patient and drugs(s) involved.

After edit checks and DURs have been performed, pharmacy system 106 can adjudicate a claim for payment with a third party 110 via communication link 109. For example, if the claim is to be paid with U.S. Government funds (e.g., government program for long-term indigent patient care), the claim can be adjudicated with the appropriate U.S. government agency or the agency's intermediary. If required, after the claim for payment has been properly adjudicated, at step 208, pharmacy system 106 creates a prescription request data packet formatted appropriately for transmission to central fill system 108. At step 210, pharmacy system 106 transmits the prescription request packet to central fill system 108.

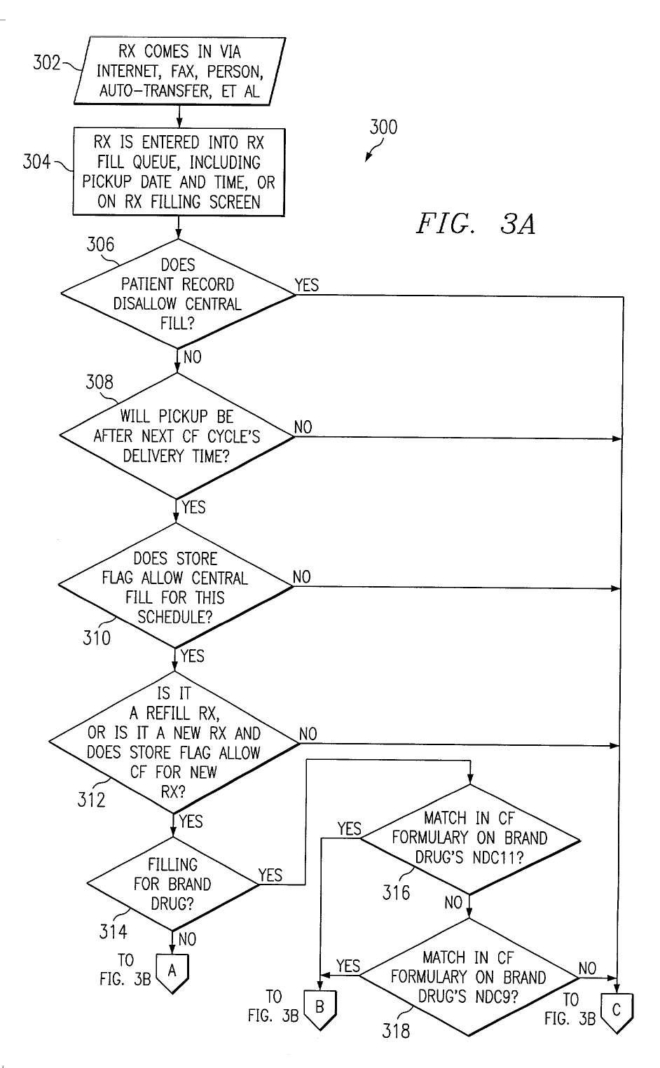

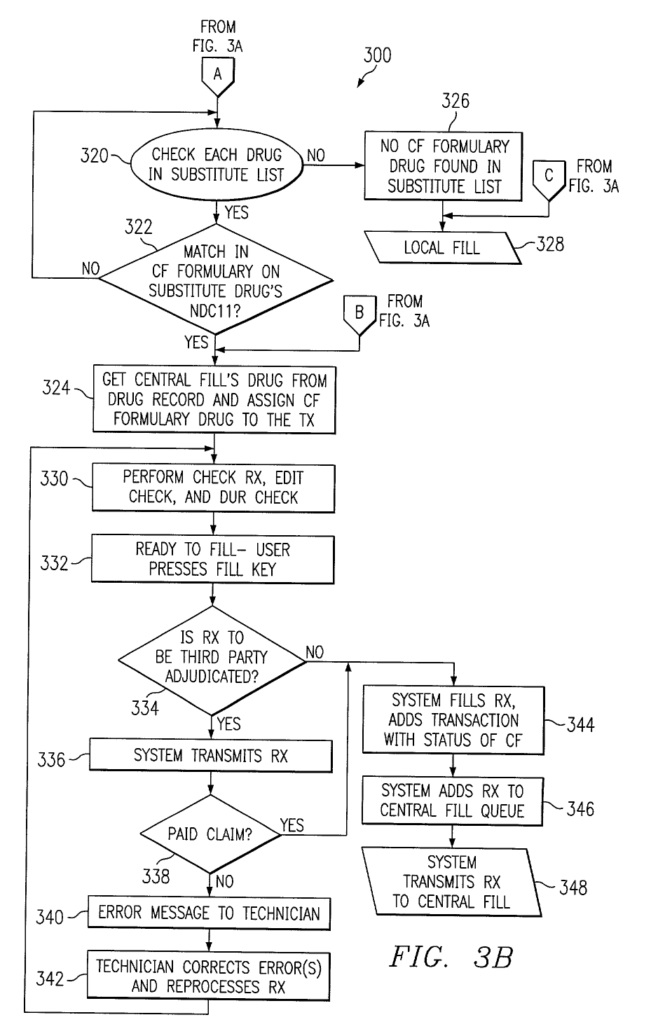

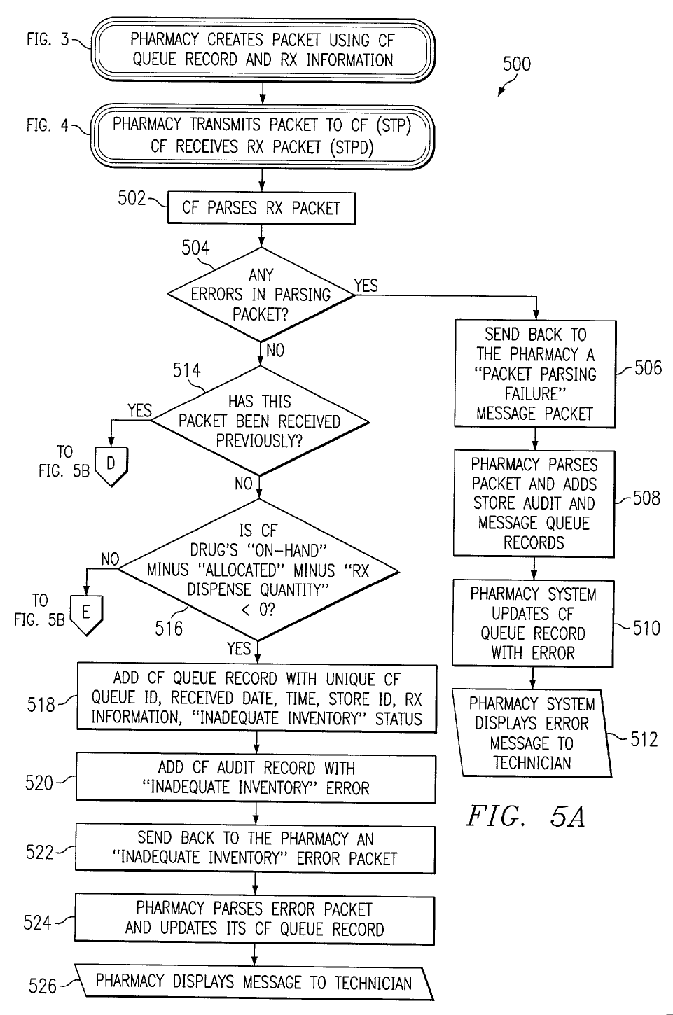

FIG. 3 is a flow diagram that illustrates an exemplary method 300 for processing prescription requests, which can be executed by a computer processor associated with pharmacy system 106 to implement an embodiment of the present invention. At step 302, a request for a new or refill prescription from a patient or prescriber is received by pharmacy system 106. As mentioned above, the request can be received via a number of different ways, such as, for example, via the Internet, facsimile, electronic transfer of data, in person from the patient, or any other appropriate way of conveying prescription information. At step 304, a technician can enter the received prescription information, including pickup date and time, into an “RX Fill Queue” screen or “Rx Filling” screen displayed on a computer monitor. At step 306, the central fill eligibility check (associated with step 206 in FIG. 2) is initiated by pharmacy system 106 determining whether the patient's record includes information that prohibits the prescription from being filled by a central fill facility. If the patient record shows that the prescription may be filled by a central fill facility, at step 308, pharmacy system 106 determines whether the prescription pickup date and time selected by the patient is subsequent to the central fill system's next scheduled delivery date and time. If so, at step 310, pharmacy system 106 determines whether the local government (e.g., State where pharmacy store is located) allows the specific drug schedule to be filled by a central fill facility. For this embodiment, a flag specified for that function can be set. If the local government allows the drug schedule to be filled by a central fill facility, at step 312, pharmacy system 106 determines whether the prescription to be filled is for a new prescription. If so, pharmacy system 106 then determines whether the local government allows new prescriptions to be filled by a central fill facility (again, for example, by determining whether a flag specified for that function is set). If new prescriptions may be filled by a central fill facility, pharmacy system 106 proceeds to step 314.

Returning to step 306, if pharmacy system 106 determines that patient 104 does not want a central fill facility to be used, then at step 328, the pharmacy system initiates a procedure to fill the prescription request from the pharmacy system's local inventory. This procedure is also initiated (step 328) if pharmacy system 106 determines (1) that the prescription pickup date and time selected by the patient is prior to the central fill system's next scheduled delivery date and time (step 308), (2) the local State government does not allow the specific drug schedule to be filled by a central fill facility (step 310), and/or (3) the local State government does not allow new prescriptions to be filled by a central fill facility (step 312).

Returning to step 314, pharmacy system 106 next determines whether the pending prescription request includes a “brand name” drug. If so, at step 316, pharmacy system 106 searches a database including a formulary available from central fill system 108, in order to determine whether the “brand name” drug's name, manufacturer, strength, and package size (referred to as National Drug Code or NDC 11) matches the name, manufacturer, strength, and package size (NDC 11) of a drug listed in the central fill system's formulary. If not, at step 318, pharmacy system 106 searches the database with the central fill system's formulary of available drugs, in order to determine whether the “brand name” drug's name, manufacturer, and strength (referred to as NDC 9) matches the name, manufacturer, and strength (NDC 9) of a drug listed in the central fill system's formulary. If not, then the pharmacy system proceeds to step 328 to initiate a procedure to fill the request from the local pharmacy inventory. Otherwise, if a match is found in the central fill system's formulary at step 316 or step 318, then the pharmacy system 106 proceeds to step 324.

Returning again to step 314, if the prescription request is not for a “brand name” drug, at step 320, pharmacy system 106 can iteratively search a database for the identity of any substitute drug authorized to fill the prescription request.

At step 322, pharmacy system 106 can also determine from the database search whether an authorized substitute drug matches a drug listed in the central fill system's 108 formulary of available drugs. If not, then returning to step 320, pharmacy system 106 checks the next drug on the substitute list for a formulary match. If no drug on the substitute list matches any drug on the central fill system's formulary, then at step 326, pharmacy system 106 initiates the local fill procedure (step 328). However, if a substitute drug is found on the central fill system's formulary, at step 324, pharmacy system 106 obtains from the database the pertinent central fill information that can be associated with the substitute drug, and assigns the appropriate drug in the central fill system's formulary as the substitute drug for the pending prescription request transaction.

Once a drug from the central fill system's 108 formulary has been identified as available for processing, at step 330, pharmacy system 106 and/or a technician performs all required edit checks, prescription checks, and DURs. After these checks have been completed satisfactorily, pharmacy system 106 considers the prescription ready to fill. Preferably, once all appropriate quality assurance procedures have been completed satisfactorily, at step 332, a technician or other user can initiate a manual procedure to have the pending prescription request filled.

At step 334, pharmacy system 106 can determine whether the pending prescription request is to be adjudicated for payment by a third party. If so, at step 336, pharmacy system 106 transmits pertinent prescription information to the appropriate third party 110 (FIG. 1) via communication link 109. For example, if the pending prescription request is to be funded by a federal program (e.g., indigent patient long-term care program), the pharmacy system can transmit the pending prescription information to the appropriate federal agency or the agency's intermediary for that program. In any event, the pending prescription request information can be conveyed to the third party by any appropriate communication medium (e.g., electronic transfer of digital files, etc.). If the pending prescription request is not to be adjudicated real-time for payment by a third party, pharmacy system 106 proceeds to step 344.

If the pending prescription information has been transmitted to a third party for adjudication, then at step 338, the pharmacy system 106 waits a predetermined amount of time for information from the third party to show that the claim for payment has been paid. If the claim is paid within the predetermined period of time, pharmacy system 106 proceeds to step 344. Otherwise, if the claim has not been paid within the prescribed period of time, then at step 340, pharmacy system 106 creates an error message for a user or technician, which indicates that the claim has not been paid. Also, this message can indicate any possible errors in the prescription or claim information that may have prohibited payment of the claim. At step 342, a user or technician can correct error(s) in the prescription information, if any, and initiate re-processing of the prescription request (step 330).

If the pending prescription request has been properly processed, at step 344, pharmacy system 106 initiates a final procedure to fill the pending prescription request. For example, pharmacy system 106 can add a transaction file for processing the pending prescription request. This file can include a flag to indicate that the transaction has a “central fill” status, and can be filled by a central fill facility. At step 346, pharmacy system 106 adds the pending prescription request to a queue of transaction files for prescriptions waiting to be filled by central fill system 108 (hereinafter referred to as a “central fill queue”) At step 348, pharmacy system 106 transmits the transaction real-time to central fill system 108 via an appropriate communication link (e.g., via a non-proprietary link 111 or proprietary link 113, 115). Note that FIG. 1 shows two paths (111 and 113, 115) for transmitting prescription request information between pharmacy system 106 and central fill system 108. As described below, prescription request information is preferably transmitted via proprietary link 113, 115 to maximize system efficiency and data throughput. However, non-proprietary link 111 is also shown to illustrate that prescription request information can be transmitted between the pharmacy system and the central fill system via any appropriate communications link which is capable of transferring relatively large data files.

An exemplary transmission path (e.g., via proprietary link 113 and 115) that can be used for conveying a queue of prescription request transaction files from pharmacy system 106 to central fill system 108 in accordance with an embodiment of the present invention, is shown in the flow diagram of FIG. 2. Also, FIG. 4 is a flow diagram that illustrates an exemplary method 400 which can be used to transmit prescription request information between pharmacy system 106 and central fill system 108 via the link 113, 115, in accordance with an embodiment of the present invention.

In order to optimize the transmission of prescription request information via link 113, 115, the following assumptions can be made: (1) The pharmacy system is preferably operating on a Transmission Control Protocol/Internet Protocol (TCP/IP) network; and (2) Prescription information can be transmitted from pharmacy system 106 to central fill system 108 in real-time (e.g., these transmissions can occur at anytime day or night). As such, referring to step 402 in FIG. 4, pharmacy system 106 preferably executes an application program that creates a packet of data containing prescription request information for transmission (e.g., packet formatted in accordance with TCP/IP). The application program then executes a Simple Transfer Protocol (STP) communications program to transmit the packet containing the prescription request information to a host system 120 via communication link 113 (step 210 in FIG. 2). Essentially, an STP platform functions as a messaging hub, and can route addresses and other information (e.g., prescription data packets) throughout a network. An STP program can also be used to transmit status update requests, formulary update requests, etc. Preferably, if pharmacy system 106 is part of a group or chain of related pharmacies, host system 120 is located at a centralized site for the group or chain (e.g., chain headquarters).

For this exemplary embodiment, host system 120 can be a PDX Host System®, which is offered by PDX Inc. as a database maintenance, administrative and communications application for pharmacy systems. In this regard, host system 120 can include a Unix Tunnel Daemon (UTD), which is an operating system, program and/or server capable of transferring large data files between system or network nodes. Notably, in a Unix operating system context, daemons are programs or processes that run in the background and attend to various tasks. For this example embodiment, a UTD can be used to perform the task of transferring data files containing prescription information between different system nodes. However, although host system 120 uses a UTD to transfer prescription-related data between systems for this embodiment, the present invention is not intended to be so limited and can include any appropriate data communications application that can adequately perform these data transfer functions.

At step 404, for this example embodiment, host system 120 routes (e.g., using a UTD) the prescription request information in data packet form to National Health Information Network (NHIN®) system 112 via communication link 113 (step 212 in FIG. 2). NHIN system 112 is, among other things, a proprietary data center that provides pharmacies and other customers in the pharmaceutical industry with a comprehensive database of up-to-date and standardized drug file information, and maintenance of third party files. For example, an NHIN database can maintain the following data files: drug files; third party plan benefit files; SIG files; physician files; drug manufacturer information files; and disease management files. For this embodiment, NHIN system 112 also includes a UTD for routing prescription request information to other system nodes. However, although NHIN system 112 uses a UTD to transfer prescription-related data between systems, the present invention is not intended to be so limited and can include any appropriate data communications application that can adequately perform these data file maintenance and transfer functions.

At step 406, NHIN system 112 routes (e.g., using a UTD) the prescription request packet to central fill system 108 via communication link 115 (step 214 in FIG. 2). At step 408, an STP Daemon (STPD) included at central fill system 108 receives the prescription request packet from NHIN system 112 (step 216 in FIG. 2). In addition to receiving prescription request packets, an STPD can also execute computer programs, and receive status update requests and formulary update requests.

In response to receiving a prescription request packet, at step 410, central fill system 108 (e.g., using an STPD) executes an application program to parse the packet and retrieve the prescription request information for further processing. Also, central fill system 108 creates a packet with appropriate response information (e.g., acknowledges that a prescription request has been received), and transmits the response packet back to NHIN system 112 (e.g., using an STPD) via link 115. At step 412, NHIN system 112 transmits the response packet to host system 120 (e.g., using a UTD) via link 113. In return, at step 414, host system 120 transmits the response packet to pharmacy system 106 (e.g., using a UTD) via link 113. At step 416, an application program at pharmacy system 106 parses the received response packet, and retrieves the response information for further processing.

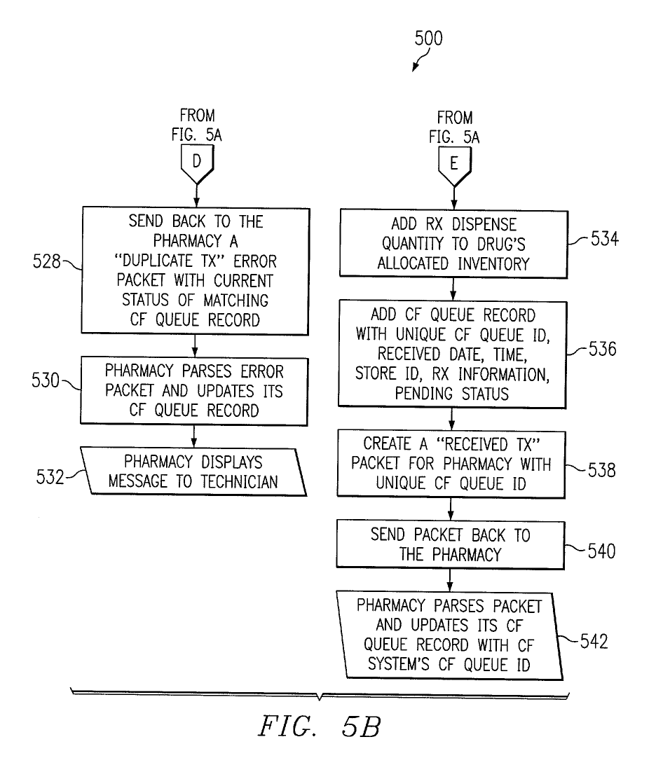

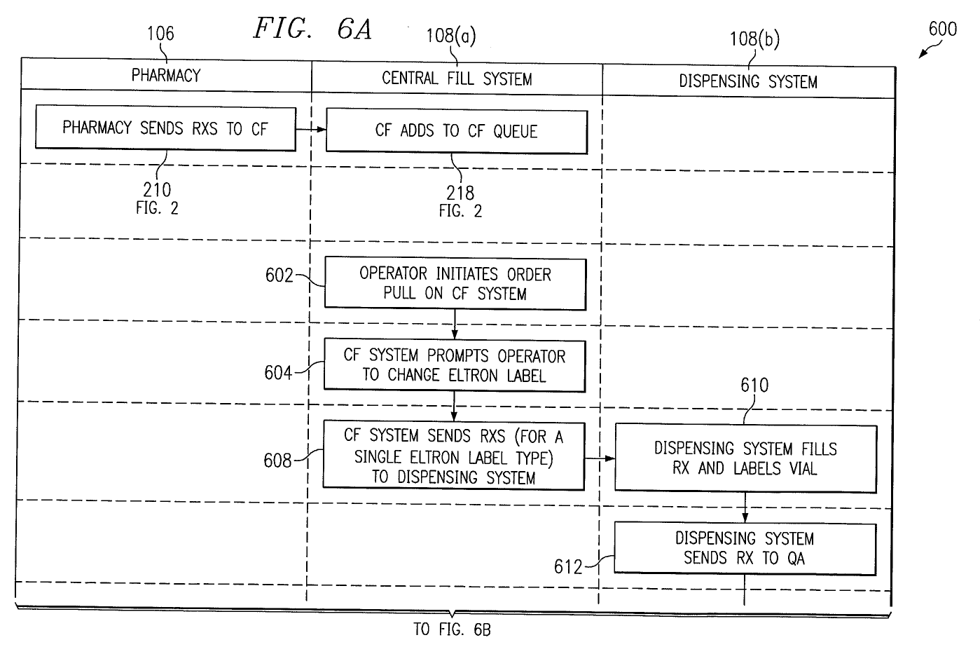

FIG. 5 is a flow diagram that illustrates an exemplary computer-implemented method 500 that can be used by central fill system 108 and pharmacy system 106 to process prescription requests, in accordance with an embodiment of the present invention. At step 502, central fill system 108 (e.g., using an appropriate application program) parses a prescription request packet received from pharmacy system 106 via an appropriate communication link 111 or 113, 115). At step 504, central fill system 108 determines whether there are any errors associated with the prescription request packet being parsed. If so, central fill system 108 creates a “packet parsing failure” message packet and transmits that packet back to pharmacy system 106 (e.g., as a response packet illustrated by FIG. 4).

If pharmacy system 106 receives a “packet parsing failure” message packet, at step 508, pharmacy system 106 parses the error message information from the received packet, and adds appropriate audit and message queue records to the error message information. Audit files can be maintained by pharmacy system 106 for pharmacy stores, in order to record errors and notifications to be reported to a pharmacy system administrator. At step 510, pharmacy system 106 updates its central fill queue record with the error message information. At step 512, pharmacy system 106 displays the error message information on a computer monitor to a user or technician.

Returning to step 504, if central fill system 108 determines that there are no errors in the prescription request packet being parsed, then at step 514, central fill system 108 determines whether this packet was previously received. If not, at step 516, central fill system 108 determines whether there is not enough inventory on-hand to fill this prescription request. If there is not enough central fill inventory on-hand, at step 518, central fill system 108 adds a central fill queue record to the transaction record being processed. The added central fill queue record includes a unique central fill queue identifier, the date and time the prescription request was received, the identity of the local pharmacy in pharmacy system 106 that originated the prescription request, the prescription request information, and an “inadequate inventory” status flag. At step 520, central fill system 108 adds a central fill audit record that includes the “inadequate inventory” error. At step 522, central fill system 108 transmits an “inadequate inventory” packet back to pharmacy system 106 (e.g., as a response message illustrated by FIG. 4). At step 524, pharmacy system 106 parses the received error packet and updates the central fill queue record accordingly to reflect the inadequate inventory error. At step 526, pharmacy system 106 displays an “inadequate inventory” error message for that prescription request to a user or technician (preferably on a computer monitor).

Returning to step 514, if the prescription request packet had been previously received, then at step 528, central fill system 108 transmits a “duplicate transmission” error packet back to pharmacy system 106 (e.g., as a response packet shown in FIG. 4). At step 530, pharmacy system 106 parses the received error packet and updates the central fill queue record accordingly. At step 532, pharmacy system 106 displays a “duplicate transmission” error message to a user or technician.

Returning to step 516, if central fill system 108 determines that there is enough inventory available on-hand to fill the prescription request, at step 534, central fill system 108 adds the quantity of the drug to be dispensed to the quantity shown in that drug's allocated inventory. At step 536, central fill system 108 adds a central fill queue record to the transaction record being processed. The added central fill queue record includes a unique central fill queue identifier, the date and time the prescription request was received, the identity of the local pharmacy in pharmacy system 106 that originated the prescription request, the prescription request information, and a “pending” status flag. At step 538, central fill system 108 creates a “received transmission” packet for transmission back to pharmacy system 106. The “received transmission” packet includes the unique central fill queue identifier for this prescription request. At step 540, central fill system 108 transmits the “received transmission” packet to pharmacy system 106 (e.g., as a response message shown in FIG. 4). At step 542, pharmacy system 106 parses the “received transmission” packet, and updates the central fill queue record with the unique central fill queue identifier received from central fill system 108. Notably, the above-described steps of creating a response packet, transmitting the response packet to pharmacy system 106, which parses and applies the information in the response packet, are also illustrated by steps 224 through 234 in FIG. 2.

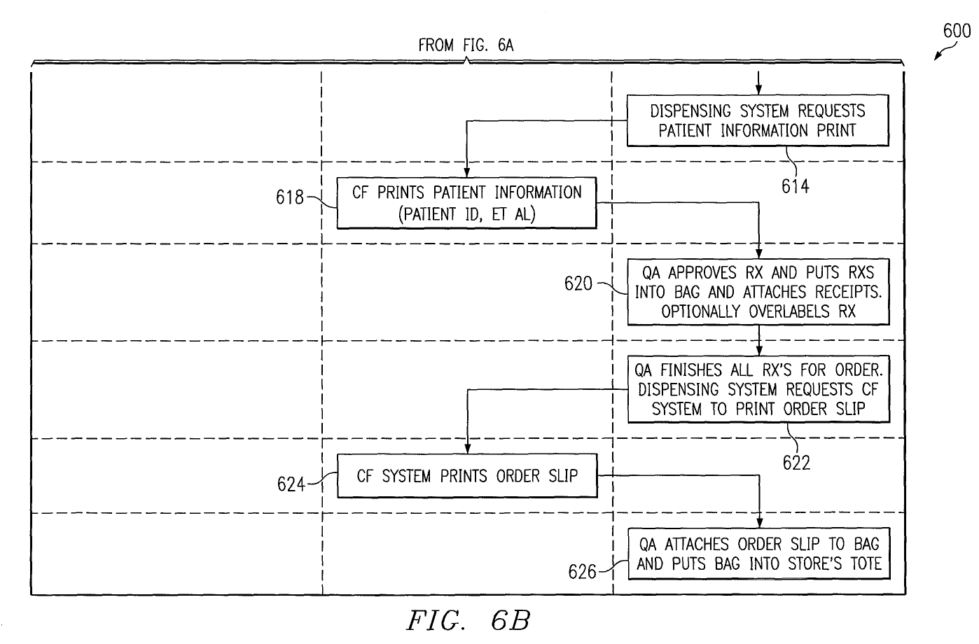

FIG. 6 is a flow diagram that illustrates an exemplary method 600 that can be used by a computer processor associated with central fill system 108 to process prescription requests, in accordance with an embodiment of the present invention. For illustrative purposes only, referring to FIG. 1, central fill system 108 includes a central fill administrative system part 108(a), and a prescription drug dispensing system part 108(b). For this embodiment, dispensing system part 108(b) maintains a central fill inventory of drugs manufactured and/or sold by one or more pharmaceutical wholesalers 118.

Preferably, central fill system part 108(a) processes prescriptions to be transmitted to dispensing system part 108(b) in a batch mode. This procedure is referred to as an “order pull”. At step 602, an operator associated with central fill system part 108(a) can initiate an “order pull” for a pending prescription request, by selecting an “Order Pull” option displayed (e.g., by a GUI) on a monitor and “pressing” a “start” function key. This procedure may also be automated to run at predetermined intervals throughout the day.

In response, central fill system part 108(a) updates the order identification field in the central fill queue to prepare the central fill queue with the pending transactions (orders to be filled) for transmission to dispensing system part 108(b). For example, a unique order ID is assigned to the associated central fill queue records, for each unique pharmacy NCPDP number and responsible party code. For this exemplary embodiment, a responsible party code is preferably a pharmacy system code that can link multiple patients to a family. Also, an order can be one or more prescriptions grouped together by responsible party (e.g., usually for a family). Central fill system part 108(a) sorts the orders in the central fill queue by: Eltron label code→store route group→store stop→store NCPDP number. If an order being processed is the final order for that day, as an option, an operator can select a range of order processing cut-off times. If such an option is selected, central fill system part 108(a) selects those pharmacy store routes for order processing with a central fill cut-off time that falls within the selected range. Central fill system part 108(a) then creates a data packet for each central fill queue record including the same order ID, and assigns the order packet a unique filename. Preferably, each order is associated with one or more related prescription requests (e.g., different prescriptions for two or more persons in the same family).

At step 604, central fill system part 108(a) prompts an operator (e.g., by displaying an appropriate message on a computer monitor) to change the Eltron label (e.g., bar-coded label), if necessary, to appropriately reflect the order to be filled. At step 608, central fill system part 108(a) transmits the order packet for each order (e.g., Eltron label type) to dispensing system part 108(b).

For example, central fill system part 108(a) can execute an STP program that transmits the order packet to a specified port at dispensing system part 108(b). If the STP program receives an acknowledgment (“ACK”) message from dispensing system part 108(b), central fill system part 108(a) updates its prescription records with a “Sent” status flag. If the STP program does not receive an “ACK” message from dispensing system part 108(b), the order packet can be re-transmitted (up to a predetermined number of times).

Essentially, an “order pull” being processed can include many different orders. Each order can include one or more prescriptions for a responsible party. Preferably, central fill system part 108(a) transmits all of the prescription requests for one order at one time to dispensing system part 108(b). After transmitting an order packet to dispensing system part 108(b), central fill system part 108(a) waits for an acknowledgment message from dispensing system part 108(b). Upon receiving an acknowledgment message for an order packet, central fill system part 108(a) transmits the next order packet to dispensing system part 108(b). This procedure is repeated until all orders within that order pull have been transmitted to dispensing system part 108(b) and acknowledged.

At step 610, upon receiving an order packet from central fill system part 108(a), dispensing system part 108(b) parses the packet and applies the prescription order information received. Preferably, dispensing system part 108(b) dispenses only complete prescription drug(s) (i.e., no prescriptions are partially filled). In other words, if there is not enough stock on hand to fill a prescription request completely, dispensing system part 108(b) sends a response message (e.g., by executing an STP program) to central fill system part 108(a) that includes a “rejection” status flag for that prescription. For prescription requests that can be filled completely, dispensing system part 108(b) prints a generic, “stock label” for that prescription, and places the labeled prescription drug in the appropriate tote for shipping. Alternatively, as an option, dispensing system 108(b) can print a prescription label with the originating pharmacy's logo. All of the prescriptions in the order can be processed in the above-described way.

At step 612, a Quality Assurance (QA) pharmacist associated with dispensing system part 108(b) can review each order being filled. For example, using a bar code scanner, a QA pharmacist can scan each prescription from an order. At step 614, dispensing system 108(b) sends an appropriate message (e.g., using an STP program) to central fill system part 108(a) which requests a printout of patient information associated with the scanned prescription. At step 618, central fill system part 108(a) sends an appropriate file for that scanned prescription to a printer, which can be accessed by the QA pharmacist involved. At step 620, the QA pharmacist retrieves the patient information from the printer, and double-checks the prescription information with the printout. If the review is successful, the QA pharmacist can approve the prescription for filling. If the prescription is approved, the QA pharmacist places the prescription drug into a bag and attaches appropriate receipts to the bag. The QA pharmacist can place an updated prescription label on the bottle. This labeling is done to match the central fill bottle label with that of the retail pharmacy.

After the QA pharmacist has approved the prescription for shipping, dispensing system part 108(b) creates a status update packet and sends it to central fill system part 108(a). Upon receiving the status update packet, central fill system part 108(a) updates the central fill queue with the prescription information from the dispensing system part 108(b).

At step 622, when all of the prescriptions in an order have been processed (e.g., filled or rejected), dispensing system part 108(b) sends a message to central fill system part 108(a) that requests the central fill system part to print an order slip. An order slip is a document that lists each prescription in an order. At step 624, central fill system part 108(a) prints an order slip that includes all of the prescriptions in that order. At step 626, a pharmacist associated with dispensing system part 108(b) places all prescriptions and patient information related to an order into a bag, and attaches the appropriate order slip to that bag. The order bag is then routed to a dispatching area for shipping to pharmacy system 106 (e.g., prescription transport or shipping route represented by link 117). For example, a QA pharmacist can place an order bag into a tote destined for delivery to a particular pharmacy store.

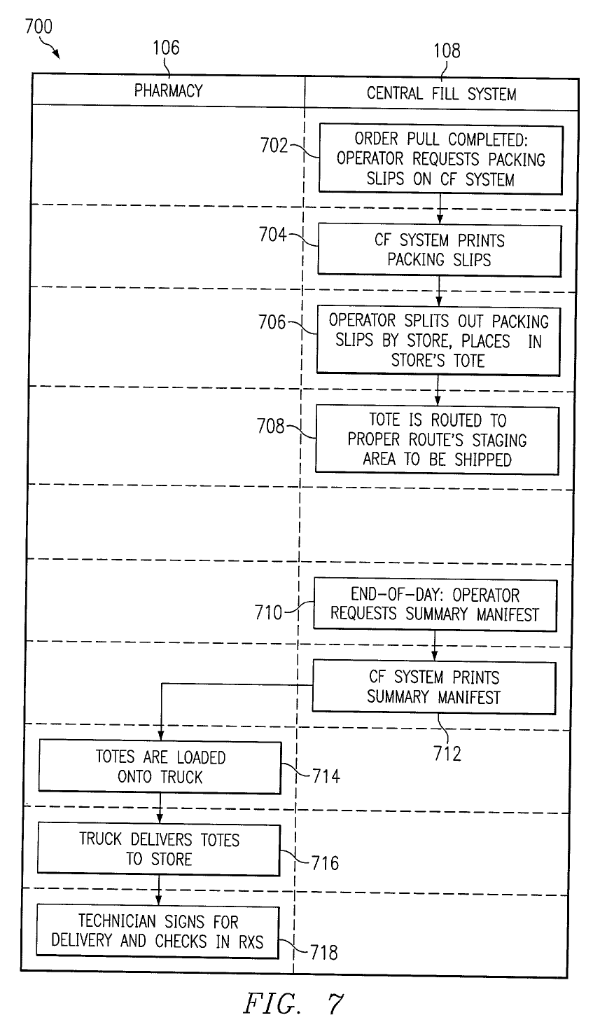

FIG. 7 is a flow diagram that illustrates an exemplary method 700 that can be used by central fill system 108 to process prescription requests, in accordance with an embodiment of the present invention. At step 702, when an operator has completed an order pull, and the order processing for that order pull has been completed, the operator can select the option “print packing slips” on a computer monitor associated with central fill system 108. The operator can then select (e.g., from a list of completed order pulls) which order pull is to have packing slips printed. A packing slip is a document that lists each prescription in a tote. The operator can then select the “start printing” function key. At step 704, in response to the operator's instructions, central fill system 108 prints packing slips for the selected order pulls.

Preferably, when printing one or more packing slips for a selected order pull, central fill system 108 selects those prescriptions in the central fill queue that have been approved by the QA pharmacist for that particular order pull. The printed report can be sorted in order by route group→route→stop. Central fill system 108 then updates the status of those prescriptions in the central fill queue to “Ready for Shipment to Pharmacy”. At step 706, an operator at central fill system 108 can separate the packing slips by store, place the appropriate packages with packing slips into the totes for the respective pharmacy stores, and seal the totes. At step 708, each tote is conveyed to an appropriate staging area for shipping to a pharmacy store.

At step 710, when all order processing has been completed for that day, and orders are ready to be shipped to the appropriate pharmacy stores, an operator can request central fill system 108 to print Summary Manifest documents (step 712). A Summary Manifest is a document that lists the stops on a shipping route and the items to be delivered at each stop. For these reports, central fill system 108 selects those prescriptions in the central fill queue that have a status of “Ready for Shipment to Pharmacy”. After these prescriptions have been selected, central fill system 108 updates the status of these prescriptions in the central fill queue as “Shipped”. At step 714, the totes can be loaded onto a truck for shipping. The truck driver can use the Summary Manifest for guidance in loading the truck and delivering the totes to the appropriate pharmacy stores. At step 716, the totes are delivered to the pharmacy stores. At step 718, a technician at pharmacy system 106 can sign the Summary Manifest and thereby acknowledge receipt of the shipped prescriptions. The technician at pharmacy system 106 then opens the tote and checks in the prescriptions filled at the central fill pharmacy by using a bar-code scanner. The pharmacy system will set those prescriptions to a status of “Processed,” which means that the prescription has been placed in the bin and is ready for patient pick-up.

Although a preferred embodiment of the method and apparatus of the present invention has been illustrated in the accompanying Drawings and described in the foregoing Detailed Description, it will be understood that the invention is not limited to the embodiment disclosed, but is capable of numerous rearrangements, modifications and substitutions without departing from the spirit of the invention as set forth and defined by the following claims.

1. A system for processing prescription requests, comprising:
a first pharmacy prescription processing subsystem; and
a central fill prescription processing subsystem coupled to the first pharmacy prescription processing subsystem and a second pharmacy subsystem by a transmission medium, the first pharmacy prescription processing subsystem operable to:
receive a plurality of prescription requests;
create a queue of prescription requests from the received plurality of prescription requests, each prescription request in the queue eligible to be filled by a central fill inventory;
convert the queue of prescription requests to a transmission format; and
transmit the converted queue of prescription requests to the central fill prescription processing subsystem by the transmission medium; and
the central fill prescription processing subsystem operable to:
receive the converted queue of prescription requests with the transmission format;
convert the queue of prescription requests from the transmission format to a processing format;
fill a plurality of prescription requests in the queue of prescription requests from the central fill inventory; and
dispense a plurality of drugs from the central fill inventory, the dispensed plurality of drugs associated with the plurality of filled prescription requests.

2. The system of claim 1, wherein the transmission medium comprises at least one Unix Tunnel Daemon.

3. The system of claim 1, wherein the transmission medium comprises at least one STP Daemon.

4. The system of claim 1, wherein the transmission format comprises a packet data format.

5. The system of claim 1, wherein the pharmacy prescription processing subsystem operates in a TCP/IP network.

6. The system of claim 1, further comprising an IVR system for entering at least one of the plurality of prescription requests to the pharmacy prescription processing subsystem.

7. The system of claim 1, further comprising:
a PDX Host system coupled to the pharmacy prescription processing subsystem; and
an NHIN system coupled to the PDX Host system and the central fill prescription processing subsystem.

8. The system of claim 1, further comprising a billing subsystem coupled to the pharmacy prescription processing subsystem, the billing subsystem operable to process a claim for payment for at least one of the plurality of prescription requests.

9. A pharmacy prescription processing system, comprising:
means for entering a prescription request; and
a processor coupled to the means for entering, the processor operable to:
receive a plurality of prescription requests;
create a queue of prescription requests from the received plurality of prescription requests, each prescription request in the queue eligible to be filled by a central fill inventory;
convert the queue of prescription requests to a transmission format; and
transmit the converted queue of prescription requests to a central fill prescription processing system that dispenses a plurality of drugs from the central fill inventory based, at least in part, on the transmitted queue of prescription requests.

10. A central fill prescription processing system, comprising:
a central fill inventory; and
a processor coupled to the central fill inventory and operable to:
receive a queue of prescription requests in a predetermined transmission format from a pharmacy system;
convert the queue of prescription requests from the predetermined transmission format to a processing format;
fill a plurality of prescription requests in the queue of prescription requests from the central fill inventory; and
dispense a plurality of drugs from the central fill inventory, the dispensed plurality of drugs associated with the plurality of filled prescription requests.

11. A pharmacy prescription processing method, comprising the steps of:
receiving a plurality of prescription requests;
creating a queue of prescription requests from the received plurality of prescription requests, each prescription request in the queue eligible to be filled by a central fill inventory;
converting the queue of prescription requests to a transmission format; and
transmitting the converted queue of prescription requests to a central fill prescription processing system that dispenses a plurality of drugs from the central fill inventory based, at least in part, on the transmitted queue of prescription requests.

12. A central fill prescription processing method, comprising the steps of
receiving a queue of prescription requests in a predetermined transmission format from a provider;
converting the queue of prescription requests from the predetermined transmission format to a processing format;
filling a plurality of prescription requests in the queue of prescription requests from a central fill inventory; and
dispensing a plurality of drugs from the central fill inventory, the dispensed plurality of drugs associated with the plurality of filled prescription requests.

13. A method for processing prescription requests comprising:
a pharmacy prescription processing subsystem:
receiving a plurality of prescription requests, at least one of the prescription requests received from a physician and at least one of the prescription requests received from a patient;
creating a queue of prescription requests from the received plurality of prescription requests, each prescription request in the queue eligible to be filled by a central fill inventory;
converting the queue of prescription requests to a transmission format;
transmitting the converted queue of prescription requests to a central fill prescription processing subsystem;
the central fill prescription processing subsystem:
receiving the converted queue of prescription requests;
converting the queue of prescription requests from the transmission format to a processing format;
filling a plurality of prescription requests in the queue of prescription requests from the central fill inventory; and
dispensing a plurality of drugs from the central fill inventory, the dispensed plurality of drugs associated with the plurality of filled prescription requests.

14. The method of claim 13, wherein the transmitting step comprises transmitting the converted queue of prescription requests with at least one Unix Tunnel Daemon.

15. The method of claim 13, wherein the transmitting step comprises transmitting the converted queue of prescription requests with at least one STP Daemon.

16. The method of claim 13, wherein the transmission format comprises a packet data format.

17. The method of claim 13, further comprising the step of entering at least one of the plurality of prescription requests to the pharmacy prescription processing subsystem with an IVR system.

18. The method of claim 13, further comprising the step of processing a claim for payment for at least one of the plurality of prescription requests.

19. A method for processing prescription requests, comprising the steps of:
entering at least a first prescription request into a queue of prescription requests to be filled;
determining if the first prescription request is eligible to be filled from a central fill inventory;
if the first prescription request is eligible to be filled from the central fill inventory, determining if the first prescription request can be filled by a brand name drug from the central fill inventory;
if the first prescription request is not eligible to be filled from the central fill inventory, assigning the first prescription request to be filled from a local inventory;
if the first prescription request can be filled by the brand name drug from the central fill inventory, assigning the brand name drug to fill the first prescription request;
if the first prescription request cannot be filled by a brand name drug from the central fill inventory, determining if a second drug from the central fill inventory can be substituted for the brand name drug;
if the first prescription request can be filled by a second drug from the central fill inventory, assigning the second drug to fill the first prescription request;
if the first prescription request cannot be filled by a second drug from the central fill inventory, assigning the first prescription request to be filled from the local inventory;
if the first prescription request has been assigned for filling from the central fill inventory, sending the prescription fill queue including at least the first prescription request to a dispensing system associated with the central fill inventory for filling.

20. The method of claim 19, further comprising the step of:
if the first prescription request has been assigned for filling from the central fill inventory, sending billing information associated with the first prescription request to a payment system.

21. The method of claim 20, if a claim for payment associated with the first prescription request is not paid by the payment system within a predetermined amount of time, further comprising generating an error message to report that the claim has not been paid.

22. The method of claim 19, wherein the step of sending the prescription fill queue including the first prescription request to the dispensing system associated with the central fill inventory for filling comprises conveying at least one data packet including the first prescription request using at least one Unix Tunnel Daemon or at least one STP Daemon.

23. The method of claim 19, wherein the prescription fill queue comprises a plurality of prescription requests, at least one of the prescription requests requested by a physician and at least one of the prescription requests requested by a patient.

24. A method for processing prescription requests, comprising the steps of:
receiving a plurality of prescription requests to be filled from a pharmacy system;
selecting at least one prescription request from the plurality of prescription requests;
determining if a central fill inventory has adequate inventory to fill the first prescription request;
if the central fill inventory has adequate inventory to fill the first prescription request, allocating a dispense quantity for the first prescription request;
if the central fill inventory has inadequate inventory to fill the first prescription request, generating an error message to report that the central fill inventory has inadequate inventory to fill the prescription request;
if a dispense quantity has been allocated for the first prescription request, dispensing the dispense quantity from the central fill inventory.

25. The method of claim 24, further comprising the steps of:
initiating an order pull for the plurality of prescription requests, the plurality including the first prescription request having an allocated dispense quantity;
generating at least one packing slip associated with the order pull; and
substantially affixing the first packing slip to a tote, the tote including the dispensed quantity from the central fill inventory, and the tote destined for a predetermined store.

26. The method of claim 24, further comprising the steps of
initiating an order pull for the plurality of prescription requests, the plurality including the first prescription request having an allocated dispense quantity; and
generating a summary manifest report including a plurality of orders associated with the order pull.

27. The method of claim 19, the first prescription request requested by a prescriber.

28. The method of claim 19, the first prescription request requested by a pharmacy customer.
